(19) United States
(12) Patent Application Publication
(10) Pub. No.: US 2021/0041411 A1
(43) Pub. Date: Feb. 11, 2021

(54) SYSTEM AND METHOD FOR MONITORING FOR THE PRESENCE OF VOLATILE ORGANIC COMPOUNDS

(71) Applicant: Computational International LLC, Newark, DE (US)

(72) Inventors: Ken Pingsheng HU, Newark, DE (US); Geoffrey Stephen ENGEL, Souderton, PA (US)

(21) Appl. No.: 16/985,893

(22) Filed: Aug. 5, 2020

Related U.S. Application Data
(60) Provisional application No. 62/883,237, filed on Aug. 6, 2019.

(51) Int. Cl. G01N 33/00; G01N 30/64; G01N 30/20

(52) U.S. Cl. CPC G01N 33/0047; G01N 30/64; G01N 2030/025; G01N 2030/642; G01N 30/20

(57) ABSTRACT

A volatile organic compound (VOC) testing system is provided that includes a plurality of valves, and a plurality of pumps. At least one of the pumps is coupled to at least one of the valves. A plurality of sensors are coupled to the pumps and at least one of the valves. The sensors detect one or more volatile organic compounds.

### CROSS-REFERENCE PARAGRAPH

The present application claims priority to U.S. Provisional Application No. 62/883,237 filed Aug. 6, 2019, the entire contents of which is incorporated herein for all purposes by this reference.

### BACKGROUND

Volatile organic compounds (VOCs) are organic chemicals that have a high vapor pressure at ordinary room temperature. This high vapor pressure results from a low boiling point, and this low boiling point causes large numbers of VOC molecules to evaporate from the compound and readily enter the surrounding air. This characteristic is referred to as volatility.

VOCs include both human-made and naturally occurring chemical compounds. Many scents and odors are the result of VOCs. However, many VOCs are dangerous to human health or can cause harm to the environment. More specifically, although harmful VOCs are not often acutely toxic, they do typically have compounding long-term adverse health effects. As such, advance knowledge, such as by regularly occurring measurements, of the presence of VOCs is an important matter for public health and safety. VOCs are also emitted from living organisms and/or subjects, including but not limited to humans, and can be used as an indicator and/or biomarker to determine the state of these living subjects, including but not limited to the health thereof.

The measurement for the presence of VOCs typically requires large devices, such as on the order of at least 2′×2′×2′, which are very expensive due, in part, to their size and complexity. Moreover, such large measurement devices generally employ the use of chromatography, which historically has necessitated temperature and moisture controlled clean room, the use of expensive consumable gases as well as specialist training equivalent to a Ph.D. degree. Needless to say, the foregoing severely limits the broad availability of VOC testing for health screening or environmental monitoring, and these limitations are exacerbated in the developing world or disadvantaged communities due to the lack of funds for the prior art devices, or nearby laboratories to which gas for testing may be transported for testing in such prior art devices.

Alternative methods in development for VOC testing are generally quite expensive, suffer from very poor accuracy, provide infrequent measurement windows, or require specialized expertise for operation and maintenance. As such, no available technologies provide accurate, inexpensive VOC testing with broad availability at a low cost, and thus potentially hazardous public health and damaging environmental conditions as well as ineffective and less accessible disease diagnostic capabilities continues.

### BRIEF SUMMARY

According to one aspect of the subject matter described in this disclosure, a volatile organic compound (VOC) testing system is provided. The VOC testing system includes a plurality of valves, and a plurality of pumps. At least one of the pumps is coupled to at least one of the valves. A plurality of sensors are coupled to the pumps and at least one of the valves. The sensors detect one or more volatile organic compounds.

According to another aspect of the subject matter described in this disclosure, a method of analyzing a gas mixture is provided. The method includes directing a sample into one of a plurality of valves. Also, the method includes detecting one or more volatile organic compounds in the sample using a plurality of sensors. The sensors are coupled to a plurality of pumps and at least one of the valves.

According to another aspect of the subject matter described in this disclosure, a system for analyzing a gas mixture is provided. The system includes an enclosure inlet and an enclosure outlet. A testing section is coupled to the enclosure inlet and the enclosure outlet. The testing section includes a plurality of valves, and a plurality of sensors being coupled to at least one of the valves. The sensors detect one or more volatile organic compounds.

Additional features and advantages of the present invention are described in, and will be apparent from, the detailed description of this disclosure.

### BRIEF DESCRIPTION OF THE DRAWINGS

The disclosure is illustrated by way of example, and not by way of limitation, in the figures of the accompanying drawings in which like reference numerals are used to refer to similar elements. It is emphasized that various features may not be drawn to scale and the dimensions of various features may be arbitrarily increased or reduced for clarity of discussion.

FIG. 1 illustrates an exemplary embodiment of a testing system for gas chromatography (GC) volatile organic compound (VOC) testing or total volatile organic compound (TVOC) testing, in accordance with some embodiments.

FIG. 2 illustrates an exemplary embodiment of a testing section 200 used in conjunction with a testing system having a GC photoionization detector (PID) sensor and a TVOC PID sensor, in accordance with some embodiments.

FIG. 3 illustrates an exemplary embodiment of a testing section used in conjunction with a testing system having two GC PID sensors, in accordance with some embodiments.

FIG. 4 illustrates an exemplary embodiment of a testing section used in conjunction with a testing system having a single GC PID sensor and a dual GC PID sensor/TVOC sensor structure.

FIG. 5 illustrates an exemplary embodiment of a testing section used in conjunction with a testing system having two GC PID sensor/TVOC sensor structures, in accordance with some embodiments.

FIG. 6 illustrates an exemplary embodiment of a testing section used in conjunction with a testing system having multiple GC PID sensors and multiple TVOC sensors, multiple ovens, in accordance with some embodiments.

FIG. 7 shows a top view of a testing system, in accordance with some embodiments.

FIG. 8 shows a bottom view of the testing system of FIG. 7, in accordance with some embodiments.

FIG. 9 illustrates an exploded view of an exemplary embodiment of an oven assembly 900, in accordance with some embodiments.

FIG. 10 illustrates an exploded view of an exemplary embodiment of a trap assembly 1000, in accordance with some embodiments.

FIG. 11 illustrates an exploded view of an exemplary embodiment of a PID housing assembly 1100, in accordance with some embodiments.

FIG. 12A illustrates a top view of an exemplary embodiment of the custom part of FIG. 11, in accordance with some embodiments; FIG. 12B illustrates a rear view of an exemplary embodiment of the custom part of FIG. 11, in accordance with some embodiments; FIG. 12C illustrates a bottom view of an exemplary embodiment of the custom part of FIG. 11, in accordance with some embodiments.

FIG. 13 illustrates of an exemplary embodiment of a pre-trap housing assembly, in accordance with some embodiments.

FIG. 14 illustrates an exemplary embodiment of a wind meter integrated with the VOC testing system, in accordance with some embodiments.

### DETAILED DESCRIPTION

The figures and descriptions provided herein may have been simplified to illustrate aspects that are relevant for a clear understanding of the herein described devices, systems, and methods, while eliminating, for the purpose of clarity, other aspects that may be found in typical similar devices, systems, and methods. Those of ordinary skill may recognize that other elements and/or operations may be desirable and/or necessary to implement the devices, systems, and methods described herein. But because such elements and operations are well known in the art, and because they do not facilitate a better understanding of the present disclosure, a discussion of such elements and operations may not be provided herein. However, the present disclosure is deemed to inherently include all such elements, variations, and modifications to the described aspects that would be known to those of ordinary skill in the art.

The terminology used herein is for the purpose of describing particular example embodiments only and is not intended to be limiting. For example, as used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc., may be used herein to describe various elements, components, regions, layers and/or sections, these elements, components, regions, layers and/or sections should not be limited by these terms. These terms may be only used to distinguish one element, component, region, layer or section from another element, component, region, layer or section. That is, terms such as “first,” “second,” and other numerical terms, when used herein, do not imply a sequence or order unless clearly indicated by the context.

Processor-implemented modules, systems and methods of testing and monitoring are disclosed herein that may provide access to and transformation of a plurality of types of digital content, including but not limited to video, image, text, audio, metadata, algorithms, interactive and document content, which track, deliver, manipulate, transform, transceive and report the accessed content. Described embodiments of these modules, systems and methods are intended to be exemplary and not limiting. As such, it is contemplated that the herein described systems and methods may be adapted and may be extended to provide enhancements and/or additions to the exemplary modules, systems and methods described. The disclosure is thus intended to include all such extensions.

The disclosure includes a system and method for monitoring for, identifying, and quantifying organic compounds, such as, for example, compounds which may be inclusive of 1 or more carbon atoms, such as VOCs, in gas environments. More particularly, the disclosure relates to a smaller, cost effective device that concentrates, identifies, and quantifies at least VOCs. Existing systems are large in footprint and highly complex, and are thus very expensive, in contrast to the disclosed embodiments.

Embodiments may provide measurements of compound identification, concentration, threat level, and so on. Such measurements may occur in a small footprint, as referenced above, and without the need for other supporting reagents, carrier gas such as Nitrogen necessary in the solutions of the prior art.

The embodiments may provide stationary or portable indoor or outdoor environmental monitoring. The embodiments may provide low-cost health screening as well as disease diagnostics, theranostics. Health screening and environmental screening may include screening for compounds that are toxic, carcinogenic, pollutants, and/or which contribute to the development of adverse environmental conditions.

The embodiments may perform gas chromatography (GC), such as using a photoionization detector (PID) sensor or the like, of prominent organic compounds in gas streams using air drawn through a filter, and using a small pump. As such, the embodiments do not necessitate the use of high purity, high cost gas cylinders. The embodiments may perform total volatile organic compound (TVOC) testing using a TVOC sensor, such as PID sensor or the like. TVOC is testing of a wide range of organic chemical compounds to simplify analysis and reporting when these are present in ambient air or emissions.

The PID sensor operates as a sensor for gas detection. Typical PID sensors may measure VOCs and other gases. PID sensors may produce real time readings, can be operated continuously, and are commonly used as sensors and/or detectors for GC or TVOC analysis. A PID sensor is a highly selective sensor when coupled with GC techniques. In known GC techniques, a carrier gas may include lower impurities than normal air. Helium and Nitrogen are often used. Thus, the need for a carrier gas is in contrast to the disclosed embodiments.

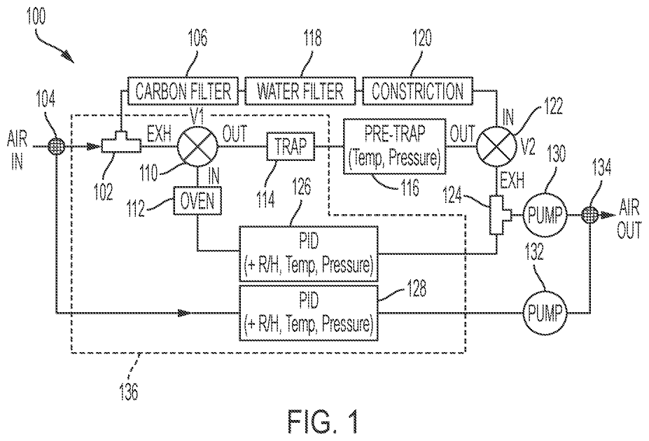

FIG. 1 illustrates an exemplary embodiment of a testing system 100 for VOC analysis or TVOC analysis, in accordance with some embodiments. As illustrated, separate channels may be provided for GC-based chemically-speciated VOC analysis, and for TVOC analysis, by way of a non-limiting example.

In particular, FIG. 1 shows testing system 100 having a tee 102 that receives at its input a sample of gas and/or air through an inlet 104 of an enclosing testing system 136. The tee 102 is connected to a carbon filter 106 and a control valve 110. Control valve 110 may receive a voltage V1 for valve control. Control valve 110 is also connected to an oven 112 and a GC trap 114. GC trap 114 is used to collect volatiles, including volatile compounds, received at its input, and is connected to a pre-trap 116.

Carbon filter 106 is connected to a water filter 118, and performs selective filtering without removing VOCs. Water filter 118 filters water from its input and is connected to a constriction 120. Constriction 120 controls the airflow through carbon filter 106 and water filter 118. Carbon filter 106 and water filter 118 can form a single filter arrangement. Control valve 122 is connected to constriction 120, pre-trap 116 and tee 124. Control valve 122 may receive a voltage V2 for valve control. Pre-trap 116 is used to measure the pressures and temperatures at one end of GC trap 114.

Oven 112 is connected to a first GC PID sensor 126 for VOC testing. First GC PID sensor 126 is connected to tee 124. A second PID sensor 128 receives the sample of gas and/or air at one input and is connected to a pump 132. The tee 124 is connected to a pump 130. Pumps 130 and 132 are connected to an outlet 134 for removal from testing system 100.

Testing system 100 includes a testing section 136 defining the section within testing system 100 that performs the various VOC and/or TVOC analysis. Testing section 136 may comprise tee 102, control valve 110, oven 112, GC trap 114 and first and second PID sensors 126 and 128. First PID sensor 126 is used for GC VOC testing while second PID sensor 128 is used for TVOC testing. In some embodiments, testing section 136 may include other arrangements of components besides those shown in FIG. 1 to be described hereinafter. Testing section 136 is coupled to both enclosure inlet 104 and enclosure outlet 134.

In some implementations, testing system 100 may include a plurality of different connections between its numerous components then those shown in FIG. 1 and still accomplish the operations described herein. In addition, tee 102 and 124 may provide inlet ports that remain perpetually open or may be manually or automatically closed. GC trap 114 may be capable of reversibly adsorbing a chemical compound, and in particular an organic compound, or more specifically a VOC.

In some embodiments, the enclosure inlet 104 includes a tee or the like. In some embodiments, the enclosure outlet 134 includes a tee or the like.

In some embodiments, the carbon filter 106 may include a charcoal filter. The charcoal filter may be formed of activated charcoal. Heaters used by GC trap 114 and oven 112 may be any suitable heater. Coolers used for cooling GC trap 114 and oven 112 may be any suitable cooler. Pumps 130 and 132 may be any suitable pump capable of generating partial vacuum for operating the system. PID sensors 126 and 128 may be any suitable sensor for sensing a chemical compound, such as a VOC.

In some cases, enclosure inlet 104 for the testing system 100 may be one or more ports going to both of the aforementioned analysis channels. Likewise, the enclosure outlet 134 from the analysis channels may comprise one or more ports from the enclosure.

In some embodiments, the system pressures may be preferably between 0 and 1 atmosphere. In some embodiments, the connections between the enclosure inlet 104 and the GC trap 114 may contain PEEK or stainless steel, and most preferably may comprise passivated stainless-steel. Other connections may use stainless steel, PEEK, FEP tubing or FEP-lined tubing.

In some implementations, the connections between the GC trap 114 and the oven 112 may contain only stainless steel.

In some embodiments, oven 112 may use one or more “cold spots” that are designed into oven 112 so as to be sufficiently long to achieve cryofocusing. The exact length may depend on the cooling mechanism used, the type of column, the thermal mass of the oven, and other factors known to the skilled artisan.

In some embodiments, heating may be achieved with low-voltage DC powered cartridge heater(s)—each of which may require a heating controller. Heating control may be capable of increasing the oven temperature at a linear rate. Oven 112 may include cartridge heater element(s) that may be controlled by a continuously variable DC voltage.

In some embodiments, the temperature of oven 112 may be controlled by heating and cooling. This temperature may be measured and controlled (via heating and cooling) in a refined manner.

In some embodiments, the temperature of GC trap 114 may likewise be controlled with heating and cooling. This temperature may be measured and controlled (via heating and cooling) in a refined manner.

The temperature of GC trap 114 may range from −10 degC to 220 degC.

In some instances, insulation may be used to protect the connections between GC trap 114 and the oven 112. If the insulation is unable to maintain the required temperatures, it may be necessary to include heating with low-voltage DC or AC powered cartridge heater(s).

Of course, the skilled artisan will appreciate the prospective need for additional circuits and controls in light of the instant disclosure. By way of example, additional controls may be included for refined control of specific heating and cooling circuits.

In some embodiments, first and second PID sensors 126 and 128 may be subjected to temperatures from −40 degC to +100 degC. This is much less than the GC trap 114 and oven 112 temperature ranges, and thus it may be necessary to thermally insulate a PID sensor from the GC trap 114 and oven heating zones.

In some implementations, first and second PID sensors 126 and 128 may operate in temperatures between −40 degC to +100 degC. In this case, first and second PID sensors 126 and 128 can be maintained in the desired temperature range, and there need not be any active heating or cooling of the first and second PID sensors 126 and 128. However, if the desired temperature range cannot be maintained, then heating or cooling may be required.

In some embodiments, relative humidity, temperature, pressure, and voltage may be monitored at a PID sensor. This may be performed in conjunction with the PID sensor readings, and therefore the RH, temperature and pressure sensor may be in the same enclosure as a PID sensor.

In some embodiments, PID humidity may be measured with +/−0.1% accuracy and 0.1% precision. PID temperature may be measured across a range of −40 degC to +100 degC with +/−0.1% accuracy and 0.1% precision. PID pressure may be measured across a range of 0 to 1 atmosphere.

In some embodiments, pressure at pre-trap 116 may be measured across a range of 0 to 1 atmosphere. The pre-trap 116 temperature may be measured across a range of −10 to 220 degC.

In some embodiments, testing system 100 may include additional pumps besides pumps 130 and 132. A single output voltage may control each of the pumps in testing system 100.

In some embodiments, testing system 100 may include additional control valves besides control valves 110 and 122. A single output voltage may control each of the control valves in testing system 100.

In some embodiments, a diagnostic port may allow an external device or screen to be used to display running status information. This diagnostic port may be RS232 or the like.

In some implementations, the data from the testing system 100 may be transmitted remotely for data capture and analysis. Data transmission may be modular, and may allow for any known transmission method, such as Bluetooth, WiFi, 4G, or the like. If data transmission halts or fails, the data can be buffered inside testing system 100, and transmitted upon restoration of the communications link. The testing system 100 may be controlled remotely.

In some instances, testing system 100 may include a real-time clock (RTC) that provides the time at which data is captured. In some embodiments, the initialization of the RTC may be done through a diagnostic port or via the time-stamp provided through a 4G transmission link or the like.

In some embodiments, a separate pressure sensor may be located inside the pre-trap 116. A separate pressure sensor may be located inside each of the PID sensor enclosures. A separate pressure sensor may be located on a controller board (in an open position) so that the current atmospheric conditions of testing system 100 can be captured.

In some embodiments, a separate humidity/temperature sensor may be located inside each of the PID sensor enclosures. A separate humidity/temperature sensor may be located inside the pre-trap 116 enclosure. A separate humidity/temperature sensor may be located on a controller board (in an open position) so that the current atmospheric conditions of the testing system 100 can be captured.

In some implementations, the enclosure for the testing system 100 may be either plastic, aluminum, stainless steel, copper, fiber glass or the like in order to keep weight down and enhance portability. The system enclosure may provide protection against dust ingress and some water resistance or waterproofing. An inlet fan can be used to draw gas and/or air into the system, such as through a baffle which prevents water ingress and which provides finger guard protection, such that the gas and/or air passes through the enclosure. An outlet fan can be used to draw gas and/or air out of the system, such as through a baffle which prevents water ingress and which provides finger guard protection, such that the gas and/or air passes out of the enclosure.

In some embodiments, the system may readily operate in various ambient temperatures and conditions, including varying humidity levels, altitudes, and atmospheric temperatures. The system may also vary as to the required power supply, such as based on numbers of elements selected as discussed above.

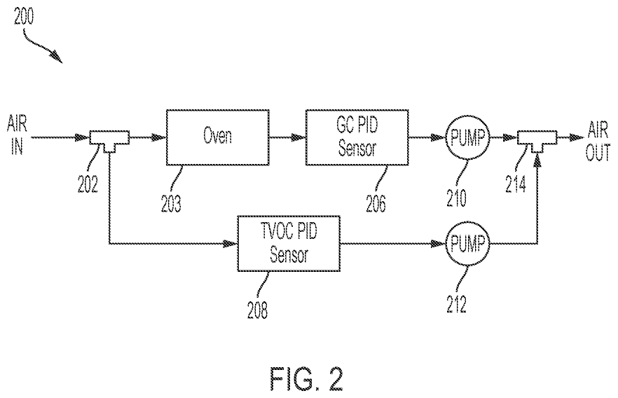

FIG. 2 illustrates an exemplary embodiment of a testing section 200 used in conjunction with testing system having a GC PID sensor and a TVOC PID sensor, in accordance with some embodiments. Testing section 200 can be used for GC VOC testing and/or TVOC testing. Testing section 200 receives a sample of gas and/or air from enclosure inlet 104. Tee 202 receives the gas and/or air from enclosure inlet 104. Tee 202 is connected to an oven 203 and a TVOC PID sensor 208. Oven 203 is connected to a GC PID sensor 206. GC PID sensor 206 is connected to a pump 210, and TVOC PID sensor 208 is connected to a pump 212. Pumps 210 and 212 are connected to a tee 214. Tee 214 directs the exhaust from the PID sensor 206 and TVOC PID sensor 208 out to enclosure outlet 134. The properties of the components of testing section 200 is similar to the properties of the components of testing section 136 of FIG. 1.

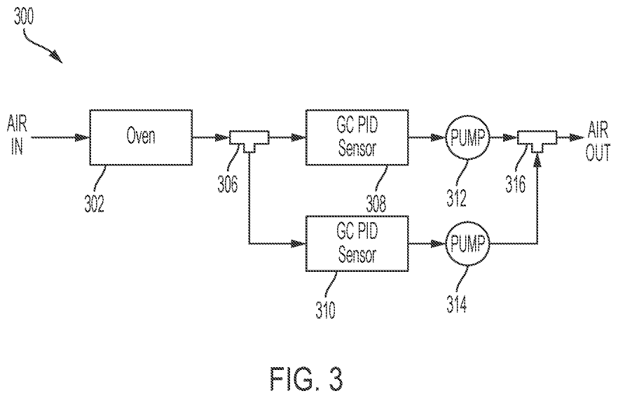

FIG. 3 illustrates an exemplary embodiment of a testing section 300 used in conjunction with a testing system having two GC PID sensors, in accordance with some embodiments. Testing section 300 can be used for dual GC VOC testing. Testing section 300 includes an oven 302 that receives a sample of gas and/or air from enclosure inlet 104. Oven 302 is also connected to a tee 306. Tee 306 is connected to GC PID sensors 308 and 310. GC PID sensor 308 is connected to a pump 312. GC PID sensor 310 is connected to pump 314. Pumps 312 and 314 are connected to a tee 316. Tee 316 directs the exhaust from GC PID sensors 308 and 310 to enclosure outlet 134. The properties of the components of testing section 200 is similar to the properties of similar components of testing section 136 of FIG. 1.

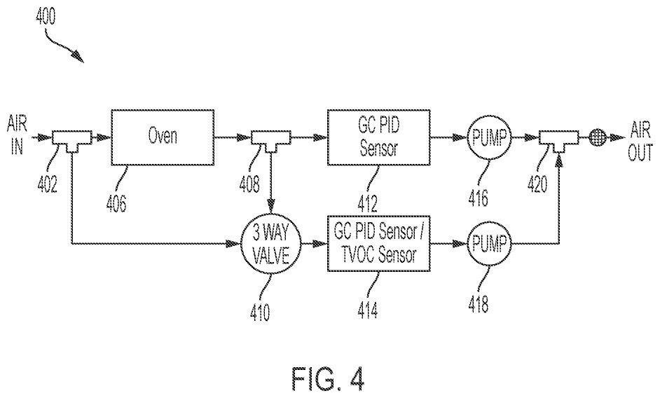

FIG. 4 illustrates an exemplary embodiment of a testing section 400 used in conjunction with testing system having a single GC PID sensor and a dual GC PID sensor/TVOC sensor structure, in accordance with some embodiments. Testing section 400 can be used for simultaneous GC VOC testing and/or dual GC VOC testing and TVOC testing. Testing section 400 includes a tee 402 that receives a sample of gas and/or air from enclosure inlet 104. Tee 402 is also connected to an oven 406 and a 3-way valve 410. Oven 406 is connected to a tee 408. Tee 408 is connected to the 3-way valve 410 and a GC PID sensor 412. The 3-way valve 410 is connected to a dual GC PID sensor/TVOC sensor structure 414. GC PID sensor 412 is connected to a pump 416, and dual GC PID sensor/TVOC sensor structure 414 is connected to a pump 418. Pumps 416 and 418 are connected to tee 420. Tee 420 directs the exhaust produced by GC PID sensor 412 and dual GC PID sensor/TVOC sensor structure 414 to enclosure outlet 134. The properties of the components of testing section 400 is similar to the properties of similar components of testing section 136 of FIG. 1.

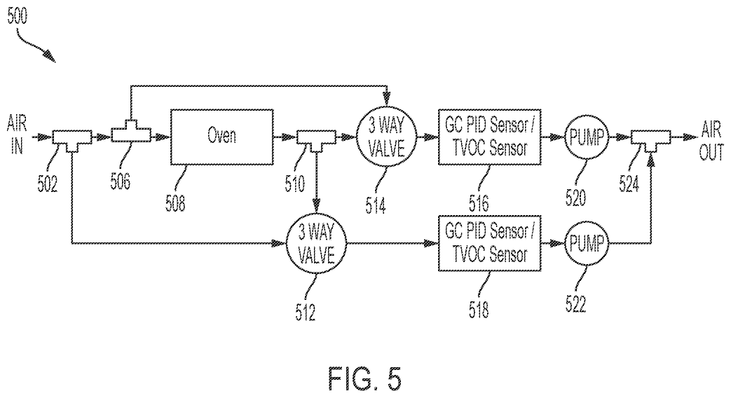

FIG. 5 illustrates an exemplary embodiment of a testing section 500 used in conjunction with testing system having two GC PID sensor/TVOC sensor structures, in accordance with some embodiments. Testing section 500 can be used for dual GC VOC testing and TVOC testing. Testing section 500 includes a tee 502 that receives a sample of gas and/or air from enclosure inlet 104. Tee 502 is also connected to a tee 506 and a 3-way valve 512. Tee 506 is connected to an oven 508 and a 3-way valve 514. Oven 508 is connected to a tee 510. Tee 510 is connected to 3-way valve 512 and 3-way valve 514. The 3-way valve 512 is connected to a first dual selectable GC PID sensor/TVOC sensor 518. The first dual selectable GC PID sensor/TVOC sensor structure 518 is connected to a pump 522. Pump 522 is connected to a tee 524. The 3-way valve 514 is connected to a second dual selectable GC PID sensor/TVOC sensor 516. The second dual selectable GC PID sensor/TVOC sensor structure 516 is connected to a pump 520. Pump 520 is connected to a tee 524. Tee 524 directs the exhaust produced by dual GC PID sensor/TVOC sensor structures 516 and 518 to enclosure outlet 134. The properties of the components of testing section 400 is similar to the properties of similar components of testing section 136 of FIG. 1.

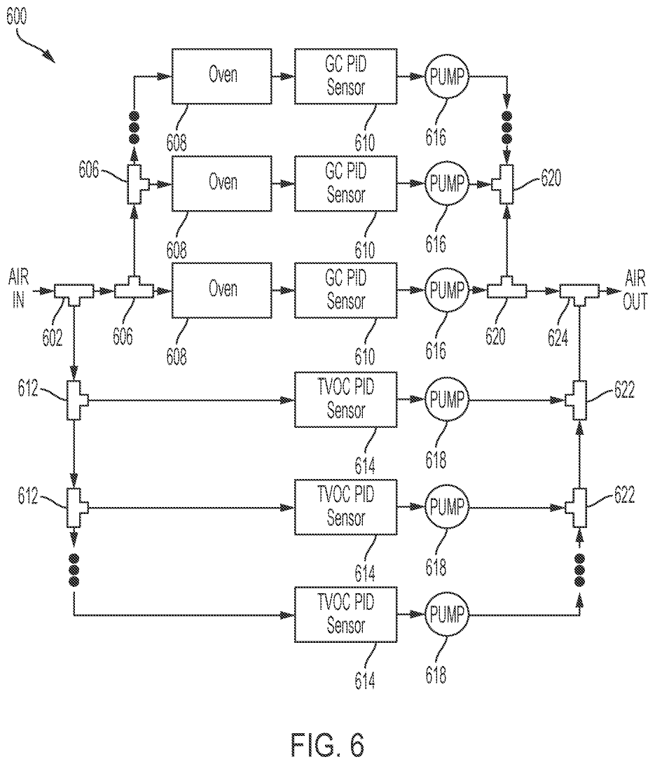

FIG. 6 illustrates an exemplary embodiment of a testing section 600 used in conjunction with testing system having multiple GC PID sensors and multiple TVOC sensors, in accordance with some embodiments. Testing section 600 can be used for multiple GC VOC testing and multiple TVOC testing. Testing section 600 includes a tee 602 that receives a sample of gas and/or air from enclosure inlet 104. Tee 602 is connected to one of a number of tees 606. Each of the tees 606 is connected to a corresponding different tee 606. Also, each of the tees 606 is connected to one of several ovens 608. Each of the ovens 608 is connected to a different GC PID sensor of multiple GC PID sensors 610. Each of the GC PID sensor 610 is connected to a different pump of multiple pumps 616. Each of the pumps 616 is connected to one of several tees 620. Moreover, each of the tees 620 is connected to a corresponding different tee 620. One of the tees 620 is connected to a tee 624.

Tee 602 is also connected to one of a number of tees 612. Each of the tees 612 is connected to a corresponding different tee 612. Also, each of the tee valves are connected to one of a number of TVOC PID sensors 614. Each of the TVOC PID sensors 614 is connected to one of many pumps 618. In addition, each of the pumps 618 is connected to one of several tees 622. Each of the tees 622 is connected to a corresponding different tee 622. One of the tees 622 is connected to tee 624. Tee 624 is used to direct the exhaust produced by GC PID sensors 610 and TVOC PID sensors 614 to enclosure outlet 134. The properties of the components of testing section 600 is similar to the properties of similar components of testing section 136 of FIG. 1.

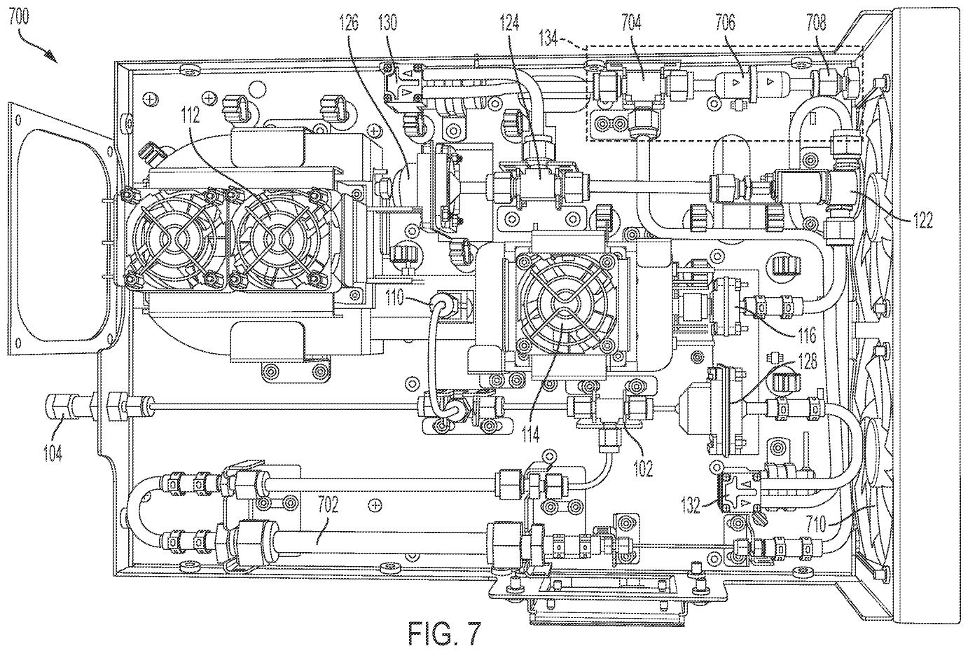

FIG. 7 shows a top view of a testing system 700, in accordance with some embodiments. Testing system 700 is substantially similar to testing system 100 and includes similar components. The same numbering applied in testing system 100 is applied to FIG. 6. Testing system 700 includes enclosure inlet 104 being connected to tee 102. Tee 102 is connected to a filter arrangement 702, which includes carbon filter 106, water filter 118, and constriction 120, as shown in FIG. 1. Also, tee 102 is connected to control valve 110. Control valve 110 is also connected to oven 112 and GC trap 114. GC trap 114 is connected to pre-trap 116. Pre-trap 116 is connected to control valve 122. Control valve 122 is connected to constriction 120 and tee 124.

PID sensor 126 is connected to tee 124 and oven 112. Also, PID sensor 126 is used for GC VOC testing. Tee 124 is connected to pump 130. PID sensor 128 is connected to enclosure inlet 104 and pump 132, and is used for TVOC testing. Pumps 130 and 132 are connected to enclosure outlet 134. In this implementation, enclosure outlet 134 may include a tee 704 that may be connected to a muffler 706. Muffler 706 is connected to an outlet 708. Outlet 708 is used to expel gas, air, and/or exhaust from testing system 700.

In some embodiments, the number of interconnections used in testing system 700 between components and/or placement of the components may change to meet particular size requirements of the enclosure. In some embodiments, the number of tees used in testing system 700 may be different and/or arranged differently as shown in FIG. 7.

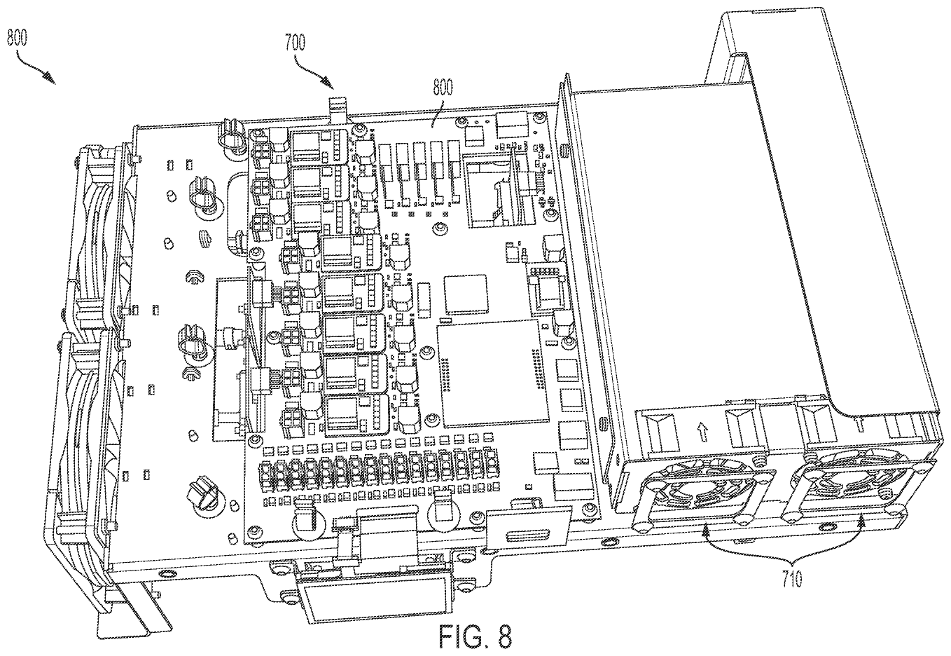

FIG. 8 is a bottom view 800 of testing system 700 of FIG. 7, in accordance with some embodiments. The bottom of testing system 700 includes a controller board 802. Controller board 802 provides the controls for most the components described herein for proper operations. Moreover, controller board 802 may allow a diagnostic port to be provided that may allow an external device or screen to be used to display running status information. This diagnostic port can be RS232 or the like. Controller board 802 may permit data from testing system 700 to be transmitted to a separate location, capturing and analyzing the data. Data transmission can be via one (1) of the following methods—allowing for transmission method to be selected before the operation: (1) Bluetooth; (b) WIFI; (c) 3G broadband communication (d) 4G broadband communication; or (e) 5G broadband communication or the like. Note if data transmission fails for whatever reason, the data can be buffered by testing system 700 using the functionalities of controller board 802, and transmitted upon restoration of the communications link.

Controller board 802 may include a real-time clock (RTC) that will provide the time at which data is captured. The initialization of this RTC can be done through the diagnostic port or via the time-stamp provided through a 4G and/or 5C transmission link or the like. The RTC should be able to achieve 10 ppm accuracy—i.e., 315 seconds per annum, as an example, but other levels of accuracy can be used. Also, controller board 802 can include the capability to detect GPS Location—and shall periodically transmit its location.

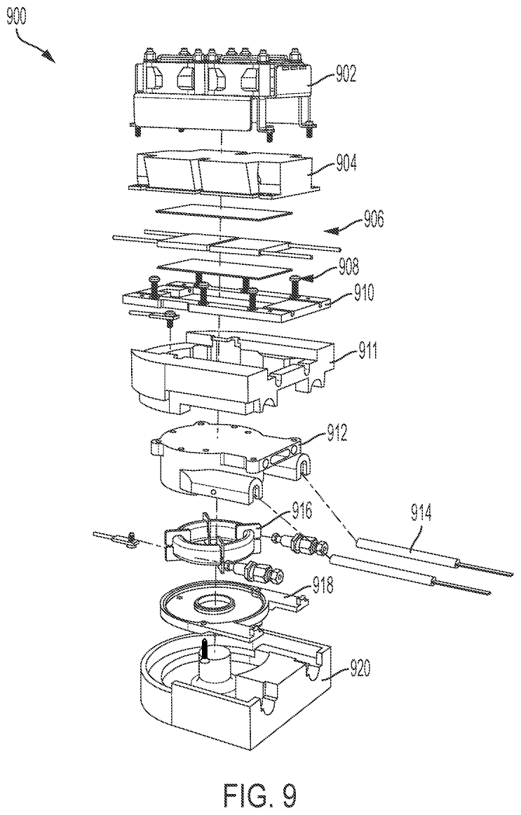

FIG. 9 illustrates an exploded view of an exemplary embodiment of an oven assembly 900, in accordance with some embodiments. The oven assembly 900 includes a cooling fan 902 used to cool the oven. A heatsink 904 is positioned beneath the cooling fan 902 to remove heat from the oven. Several thermo-electric cooling (TEC) devices 906 are used to provide additional cooling to oven assembly 900 and heatsink 904. TEC devices 906 are positioned in the middle of a first insulation spacer 910. First insulation spacer 910 is positioned in a second insulation spacer 912 using screws 908. Oven housing 912 includes heating elements 914. A toroid tubing 916 is placed between oven housing 912 and an enclosure 918 to contained heat generated by oven assembly 900. Enclosure 918 includes heat resistant material for temperature control. Enclosure 918 is positioned in a heat resistant enclosure 920. Heat resistant enclosure 920 includes screws 922 to hold the entire oven assembly 900 together.

Oven assembly 900 incorporates heating elements 914 and TEC devices 906 so that it can control its temperature from −10 degC to 220 degC. Oven housing 912 incorporates slots to hold the tubing interconnections, and ensure that the internal tubing is well constrained. An “inlet” tubing initially passes through a narrow slot to provide greater control of the temperature of the “inlet” tubing. This narrow slot is angled to provide a smooth transition path onto the outer wall of oven housing 912. Channels are included in the oven housing 912 to allow for support brackets that can constrain toroid tubing. This allows for the entire cavity to be filled with thermal putty to ensure that the temperature of all the tubing is well controlled.

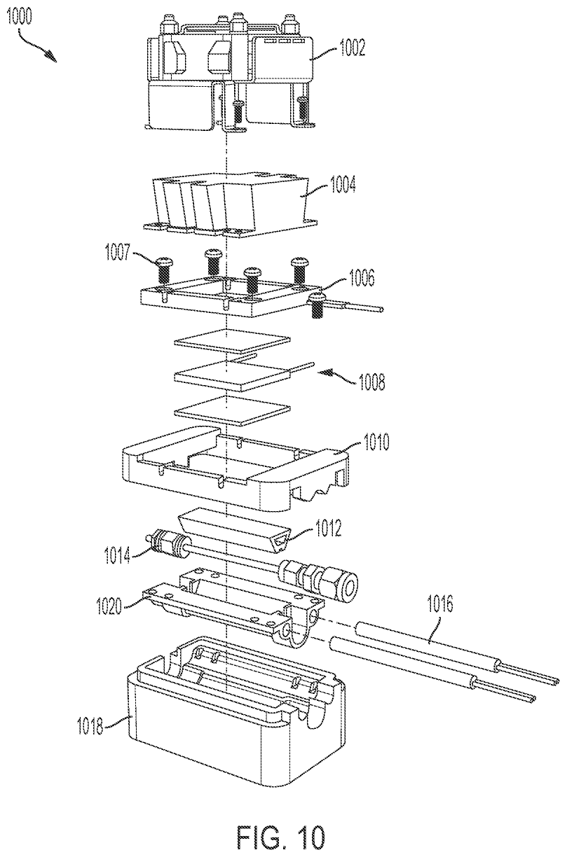

FIG. 10 illustrates an exploded view of an exemplary embodiment of a trap assembly 1000, in accordance with some embodiments. The trap assembly 1000 includes a cooling fan 1002 used to cool trap assembly 1000. A heatsink 1004 is positioned beneath the cooling fan 1002 to remove heat from trap assembly 1000. A first TEC spacer 1006 is positioned beneath heatsink 1004. Several TEC pads 1008 are positioned between first TEC spacer 1006 and a second TEC spacer 1010. Screws 1007 connect first TEC spacer 1006, TEC pads 1008, and second TEC spacer 1010 to form TEC devices. A trap tube clamp 1012 is positioned between second TEC spacer 1010 and trap housing 1014. Trap housing 1014 includes heating element 1016. Also, trap tube clamp 1012 and trap housing 1014 are positioned in an enclosure 1018. In some embodiments, enclosure 1018 may include insulation.

Trap assembly 1000 utilizes heating elements 1016 and TEC devices to control the temperature from −10 degC to 220 degC. Trap assembly 1000 can include a tightly held external diameter tube 1014 requiring trap housing 1020 and tube clamp 1012 (ie. 2-piece design to hold tube 1014). Also, there are two tubes to hold the heating elements 1016.

Enclosure 1018 encloses structures 1012 and 1020 within a thermal insulation to assist in the control of the temperature of trap assembly 1000. This insulation surrounds part of structure 1012 and 1020 except above the TEC devices.

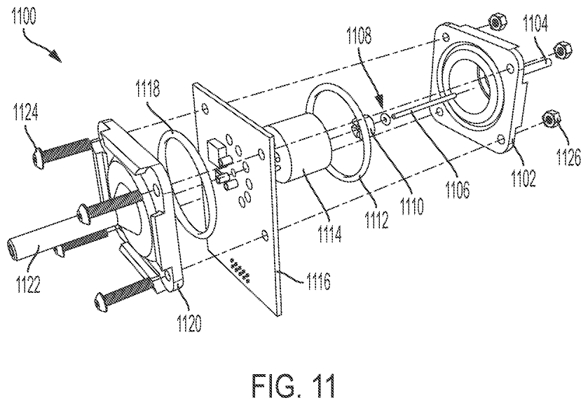

FIG. 11 illustrates an exploded view of an exemplary embodiment of a PID housing assembly 1100, in accordance with some embodiments. PID housing assembly 1100 includes a bottom housing 1102, top housing 1122, tubing 1106, gasket rings 1108, 1112 and 1118, sensor 1114, and custom printed circuit board 1116. Screws 1124 and bolts 1126 hold PID housing assembly 1100 together from top housing 1120 to bottom housing 1102. PID housing assembly 1100 includes a bottom housing 1102 that connects to an external diameter tubing 1104. Tubing 1106 which slides inside bottom housing 1102. Small gasket ring 1108 is positioned between tubing 1106 and a custom part 1110. A first large gasket ring 1112 is positioned between the custom part 1110 and a sensor 1114. Sensor 1114 is plugged into a custom printed circuit board 1116. A second large gasket 1118 is positioned between custom printed circuit board 1116 and a top housing 1120. Top housing 1120 connects to an external diameter tubing 1122.

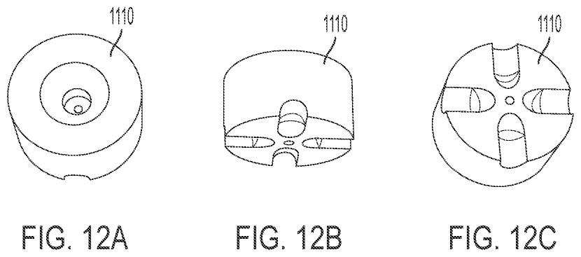

FIG. 12A illustrates a top view of an exemplary embodiment of custom part 1110 of FIG. 11, in accordance with some embodiments. FIG. 12B illustrates a rear view of an exemplary embodiment of custom part 1110 of FIG. 11, in accordance with some embodiments. FIG. 12C illustrates a bottom view of an exemplary embodiment of custom part 1110 of FIG. 11, in accordance with some embodiments. Custom part 1110 interfaces with tubing 1106 to the top of sensor 1114, as shown in FIG. 11.

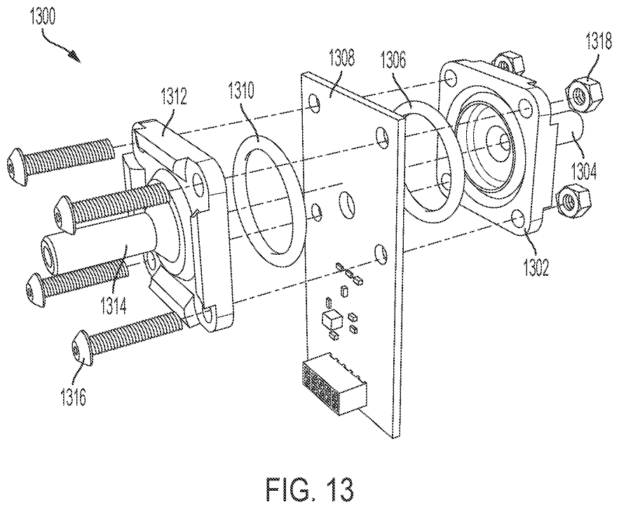

FIG. 13 illustrates an exemplary embodiment of a pre-trap housing assembly 1300, in accordance with some embodiments. Pre-trap housing assembly 1300 includes a bottom housing 1302, a top housing 1312, two gasket rings 1306 and 1310, and custom printed circuit board 1308. Screws 1316 and bolts 1318 hold pre-trap housing assembly 1300 together from top housing 1312 to bottom housing 1302. Pre-trap housing assembly 1300 includes a bottom housing 1302 that connects to an external diameter tubing 1304. A first gasket ring 1306 is positioned between bottom housing 1302 and a custom printed circuit board 1308. A second gasket ring 1310 is position between top housing 1312 and custom printed circuit board 1308. Top housing 1312 connects to an external diameter tubing 1314. Screws 1316 and bolts 11318 hold pre-trap housing assembly 1300 together from top housing 1316 to bottom housing 1302.

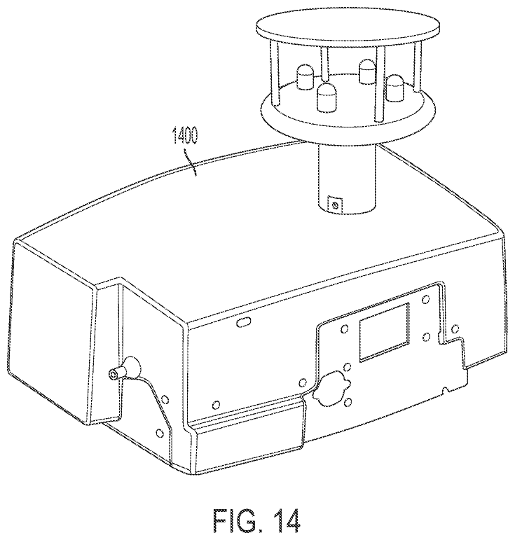

FIG. 14 illustrates an exemplary embodiment of a wind meter 1400 connectively associated with an embodiment of a housing for testing system 100. FIG. 14 is illustrative of an ultrasonic wind meter being integrated with a VOC detection monitor. Wind meter 1400 may be externally or internally connected and/or integrated to a VOC detection monitor. For example, the wind meter's operation, data and power may be coextensively controlled, and/or may share data transmission capabilities with, the VOC detection monitor's control, power, and communication systems.

Reference in the specification to “one implementation” or “an implementation” means that a particular feature, structure, or characteristic described in connection with the implementation is included in at least one implementation of the disclosure. The appearances of the phrase “in one implementation,” “in some implementations,” “in one instance,” “in some instances,” “in one case,” “in some cases,” “in one embodiment,” or “in some embodiments” in various places in the specification are not necessarily all referring to the same implementation or embodiment.

Finally, the above descriptions of the implementations of the present disclosure have been presented for the purposes of illustration and description. It is not intended to be exhaustive or to limit the present disclosure to the precise form disclosed. Many modifications and variations are possible in light of the above teaching. It is intended that the scope of the present disclosure be limited not by this detailed description, but rather by the claims of this application. As will be understood by those familiar with the art, the present disclosure may be embodied in other specific forms without departing from the spirit or essential characteristics thereof. Accordingly, the present disclosure is intended to be illustrative, but not limiting, of the scope of the present disclosure, which is set forth in the following claims.

What is claimed is:

1. A volatile organic compound (VOC) testing system, comprising:
a plurality of valves;
a plurality of pumps, at least one of the pumps being coupled to at least one of the valves; and
a plurality of sensors being coupled to the pumps and at least one of the valves, wherein the sensors detect one or more volatile organic compounds.

2. The VOC testing system of claim 1, further comprising an oven coupled to one of the valves and a first sensor of the plurality of sensors.

3. The VOC testing system of claim 1, wherein the first sensor performs gas chromatography (GC) VOC testing.

4. The VOC testing system of claim 3, wherein the first sensor is a GC PID sensor.

5. The VOC testing system of claim 1, wherein the sensors comprise at least one total VOC (TVOC) sensor performing TVOC testing.

6. The VOC testing system of claim 1, wherein the sensors comprise at least one dual GC PID sensor/TVOC sensor arrangement performing GC testing or TVOC testing.

7. A method of analyzing a gas mixture comprising:
directing a sample into one of a plurality of valves; and
detecting one or more volatile organic compounds in the sample using a plurality of sensors, wherein the sensors are coupled to a plurality of pumps and at least one of the valves.

8. The method of claim 7, further comprising an oven coupled to one of the valves and a first sensor of the plurality of sensors.

9. The method of claim 8, wherein the first sensor performs gas chromatography (GC) VOC testing.

10. The method of claim 9, wherein the first sensor is a GC PID sensor.

11. The method of claim 7, wherein the sensors comprise at least one total VOC (TVOC) sensor performing TVOC testing.

12. The method of claim 7, wherein the sensors comprise at least one dual GC PID sensor/TVOC sensor arrangement performing GC testing or TVOC testing.

13. A system for analyzing a gas mixture comprising:
an enclosure inlet;
an enclosure outlet;
a testing section coupled to the enclosure inlet and the enclosure outlet, the testing section comprising:
a plurality of valves; and
a plurality of sensors being coupled to at least one of the valves, wherein the sensors detect one or more volatile organic compounds.

14. The system of claim 13, wherein the testing section comprises an oven coupled to one of the valves and a first sensor of the plurality of sensors.

15. The system of claim 14, wherein the first sensor performs gas chromatography (GC) VOC testing.

16. The VOC testing system of claim 15, wherein the first sensor is a GC PID sensor.

17. The VOC testing system of claim 15, wherein the sensors comprise at least one total VOC (TVOC) sensor performing TVOC testing.

18. The system of claim 15, wherein the sensors comprise at least one dual GC PID sensor/TVOC sensor arrangement performing GC testing or TVOC testing.

19. The system of claim 15 further comprising a filter arrangement coupled to the testing section.
